(19) United States
(12) Patent Application Publication
(10) Pub. No.: US 2026/0086077 A1
(43) Pub. Date: Mar. 26, 2026

(54) FECAL GAS COMPONENT RATIO ANALYSIS METHOD FOR BRISTOL STOOL TYPE CLASSIFICATION

(71) Applicant: HEM PHARMA INC., Pohang-si (KR)

(72) Inventors: Yo Sep JI, Suwon-si (KR); Eun Sung JUNG, Suwon-si (KR); Young Chun PARK, Pohang-si (KR); Ju Young PARK, Pohang-si (KR)

(21) Appl. No.: 19/407,179

(22) Filed: Dec. 3, 2025

Related U.S. Application Data
(63) Continuation of application No. PCT/KR2024/007773, filed on Jun. 7, 2024.

(30) Foreign Application Priority Data
Jun. 7, 2023 (KR) 10-2023-0073105

(51) Int. Cl. G01N 33/00; G01N 33/497

(52) U.S. Cl. CPC G01N 33/0067; G01N 33/4975

(57) ABSTRACT

Provided is a fecal gas analysis method for Bristol stool type classification and body condition analysis, and more particularly, to a fecal gas component ratio analysis method capable of classifying Bristol stool types and analyzing body conditions based on the component ratio of each fecal gas. The fecal gas component ratio analysis method for Bristol stool type classification and body condition analysis enables classification of Bristol stool types regardless of measurement time, subject, diet, age, or stool weight, and also allows identification of an individual's dietary habits. Therefore, the analysis method can be applied to personal health monitoring based on Bristol stool type classification.

### TECHNICAL FIELD

The present disclosure relates to a fecal gas analysis method for Bristol stool type classification and body condition analysis, and more particularly, to a fecal gas component ratio analysis method capable of classifying Bristol stool types and analyzing body conditions based on the component ratio of each fecal gas. The present application claims the benefit of priority to Korean Patent Application No. 10-2023-0073105 filed on Jun. 7, 2023 and incorporated herein by reference in its entirety.

### BACKGROUND

Due to changes in dietary habits and various stress factors in modern life, gastrointestinal diseases are on the rise. In order to determine the progression and causes of such gastrointestinal diseases, it is important to check the condition of stool, which is the result of daily food digestion.

Although it would be ideal for an individual to directly observe the condition of their stool after defecation, many people are reluctant to do so, and it is difficult for an individual to consistently monitor information, such as defecation time, frequency, and odor.

Another method for checking the condition of stool is to use a fecal gas. In this case, the absolute concentration of gases emitted directly from the stool may be measured by one or more gas sensors to assess the condition of the stool. However, the absolute gas concentration values vary depending on measurement conditions and environmental factors, which results in reduced accuracy.

As examples of techniques developed for fecal gas analysis, an analysis system, a fecal odor gas analysis system, and an exhaled gas analysis system have been disclosed (Japanese Patent Laid-Open Publication No. 2020-187074). However, in relation to fecal gas analysis, a need remains for more accurate and effective analytical methods through further development and diverse research.

Meanwhile, the concentration of fecal gas is highly time-sensitive, and, thus, it decreases over time and changes significantly depending on the stool amount, type, and the measurement environment. Conventional fecal gas analysis methods collect stool samples in a restroom, measure gas concentrations in a laboratory, and then analyze the results. Since these methods do not measure gas concentrations immediately after stool collection, it is difficult to perform accurate analysis based solely on absolute measurement values.

Accordingly, the present inventors have made extensive efforts to develop a fecal gas analysis method that overcomes the disadvantages of the prior art. As a result, they have completed the present disclosure by discovering a fecal gas component ratio analysis method capable of accurately classifying Bristol stool types and determining health conditions with minimal influence from environmental factors, such as time or stool weight. In the discovered method, when measured values (ppm) obtained from sensors analyzing each fecal gas are converted into component ratios (%), the resulting values remain relatively constant over time.

### DISCLOSURE OF THE INVENTION

### Problems to be Solved by the Invention

The present disclosure relates to a fecal gas analysis method for Bristol stool type classification and body condition analysis, and is conceived to provide a fecal gas component ratio analysis method capable of classifying a Bristol stool type and analyzing body conditions based on the component ratio of each fecal gas.

However, the problems to be solved by the present disclosure are not limited to the above-described problems. Although not described herein, other problems to be solved by the present disclosure can be clearly understood by a person with ordinary skill in the art from the following descriptions.

### Means for Solving the Problems

A first aspect of the present disclosure provides a fecal gas component ratio analysis method for Bristol stool type classification.

### Effects of the Invention

According to an embodiment of the present disclosure, the fecal gas component ratio analysis method for Bristol stool type classification and body condition analysis enables classification of Bristol stool types regardless of measurement time, subject, diet, age, or stool weight, and also allows identification of an individual's dietary habits. Therefore, the analysis method can be applied to personal health monitoring based on Bristol stool type classification.

### BRIEF DESCRIPTION OF THE DRAWINGS

FIG. 1 is a diagram illustrating the Bristol stool scale.

FIG. 2A to FIG. 2D are diagrams illustrating gas concentration-time graphs based on outputs from eleven sensors and a total gas concentration graph (FIG. 2D) obtained by summing the real-time measurement values from the eleven sensors when gas from a single stool sample (Type 6 stool of subject C, 78 g) was measured.

FIG. 2E to FIG. 2G are diagrams illustrating component ratio-time graphs based on outputs from the eleven sensors when gas from a single stool sample (Type 6 stool of subject C, 78 g) was measured.

FIG. 3A to FIG. 3C are diagrams illustrating gas concentration-time graphs based on outputs from seven sensors and a total gas concentration graph (FIG. 3C) obtained by summing the real-time measurement values from the seven sensors when gas from a single stool sample (Type 4 stool of subject A, 22.4 g) was measured.

FIG. 3D to FIG. 3E are diagrams illustrating component ratio-time graphs based on outputs from the eleven sensors when gas from a single stool sample (Type 4 stool of subject A, 22.4 g) was measured.

FIG. 4A is a diagram illustrating a fecal gas concentration graph (ppm) for an H₂S sensor and a total concentration graph (ppm) for a sensor set.

FIG. 4B is a diagram illustrating a real-time component ratio graph (%) for the H₂S sensor.

FIG. 5A is a diagram illustrating a fecal gas concentration graph (ppm) for a C₂H₄ sensor and a total concentration graph [ppm] of a sensor set.

FIG. 5B is a diagram illustrating a real-time component ratio graph (%) for the C₂H₄ sensor.

FIG. 6A to FIG. 6F are diagrams comparing real-time component ratio graphs for respective stool types (Type 2, Type 4, and Type 6).

FIG. 6G to FIG. 6L are diagrams comparing real-time component ratio graphs for respective stool types (Type 2, Type 4, and Type 6) after reanalysis in which NH₃ was excluded.

FIG. 7 is a diagram illustrating a component ratio bar graph for a CH₂O sensor (data of subject A).

FIG. 8 is a diagram illustrating a component ratio bar graph for an NO₂ sensor (data of subjects A, B, C, and D).

FIG. 9 is a diagram illustrating a component ratio bar graph for an NH₃ sensor (data of subject A).

FIG. 10 is a diagram illustrating a component ratio bar graph for an H₂S sensor (data of subject A).

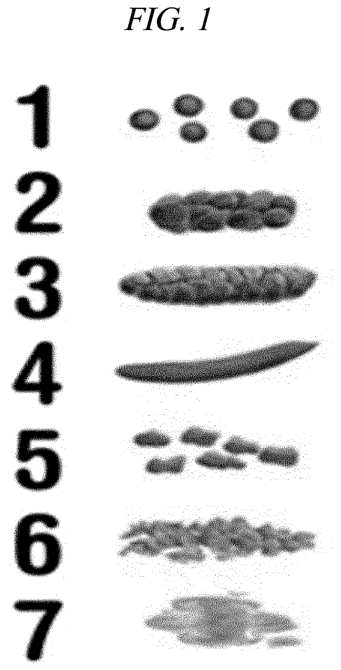

### BEST MODE FOR CARRYING OUT THE INVENTION

Hereafter, examples will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts throughout the whole document.

Throughout the whole document, the term “on” that is used to designate a position of one component with respect to another component includes both a case that the one component is adjacent to the other component and a case that any other component exists between these two components.

Further, throughout the whole document, the term “comprises or includes” and/or “comprising or including” used in the document means that one or more other components, steps, operation and/or existence or addition of elements are not excluded in addition to the described components, steps, operation and/or elements unless context dictates otherwise. Throughout the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Throughout the whole document, the term “step of” does not mean “step for.”

Throughout the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Throughout the whole document, a phrase in the form “A and/or B” means “A or B, or A and B.”

Hereafter, embodiments and examples of the present disclosure will be described in detail with reference to the accompanying drawings. However, the present disclosure may not be limited to the following embodiments, examples, and drawings.

A first aspect of the present disclosure provides a fecal gas component ratio analysis method for Bristol stool type classification.

According to an embodiment of the present disclosure, a fecal gas component ratio analysis method for Bristol stool type classification and body condition analysis includes: a process (a) of measuring a fecal gas concentration value (ppm) using a gas sensor; a process (b) of deriving a real-time component ratio graph for each gas based on the measured gas concentration value; a process (c) of obtaining a component ratio value from the derived real-time component ratio graph; a process (d) of deriving a graph using the obtained component ratio value; and a process (e) of classifying a Bristol stool type of feces by comparing the derived graph with a component ratio graph for each Bristol stool type.

According to an embodiment of the present disclosure, the component ratio analysis method is a technique for monitoring human health based on fecal gas data. The method enables determination of a subject's body constitution, health condition, or presence of disease by analyzing gas measurement values (ppm) obtained from each of eleven gas sensors in a sensor array as component ratios relative to a total gas concentration graph derived from the measurement values of the eleven gas sensors.

The concentration of fecal gas is highly time-sensitive, and, thus, it decreases over time and changes significantly depending on the stool amount, type, and the measurement environment. Therefore, it is difficult to perform analysis for monitoring health condition based solely on absolute measurement values (ppm). However, according to the component ratio analysis method of the present disclosure, it was confirmed that when gas concentration-time graphs (ppm) based on outputs from the respective sensors were converted into component ratio-time graphs (%) (real-time component ratio graphs), the resulting values remain relatively constant over time, regardless of the measurement time point after stool collection, as compared with the gas concentration measurement values (ppm).

According to an embodiment of the present disclosure, the gas sensor used in the process (a) may include one or more selected from the group consisting of NH₃, CO, H₂, VOC, NO, NO₂, H₂S, SO₂, C₂H₄, CH₂O and ETO gas sensors, but is not limited thereto. The analysis method may also be effectively applied to any sensor combination composed of a plurality of sensors (including a partial combination of the eleven sensors or other sensor combinations not mentioned herein) to monitor and determine human body condition.

According to an embodiment of the present disclosure, the process (b) may be performed as shown in Equations 1 and 2 below:

$${ex\_sensor}_{Ratio} ( i ) [ \% ] = \frac{{ex\_sensor}_{ppm ( i )}}{{Total}_{ppm ( i )}} \times 100 \qquad [ Equation 1 ]$$

$$i = Data Number , {ex\_sensor}_{Ratio} = \begin{matrix}COMPONENT RATIO MEASURED BY A SPECIFIC SENSOR \\ FOR SEPARATE ANALYSIS\end{matrix}$$

$${ex\_sensor}_{ppm} = CONCENTRATION MEASURED BY A SPECIFIC SENSOR FOR SEPARATE ANALYSIS ,$$

$${Total}_{ppm} = TOTAL SUM OF GAS CONCENTRATIONS MEASURED BY ELEVEN SENSORS$$

$${Sensor}_{Ratio} ( i ) [ \% ] = \frac{{Sensor}_{ppm} ( i )}{no\_ex \_sensor {\_Total}_{ppm ( i )}} \times 100 \qquad [ Equation 2 ]$$

$$i = Data Number , {sensor}_{Ratio} = \begin{matrix}COMPONENT RATIOS OF GASES MEASURED BY \\ THE OTHER TEN SENSORS EXCLUDING ex\_sensor\end{matrix}$$

$${sensor}_{ppm} = GAS CONCENTRATIONS MEASURED BY THE OTHER SENSORS EXCLUDING ex\_sensor ,$$

$$no\_ex \_sensor {\_Total}_{ppm} = \begin{matrix}TOTAL SUM OF GAS CONCENTRATIONS MEASURED BY \\ THE OTHER TEN SENSORS EXCLUDING ex\_sensor\end{matrix}$$

Desirably, the process (b) may be performed as shown in Equations 3 and 4, but is not limited thereto.

$${NH}_{{3}_{Ratio}} ( i ) [ \% ] = \frac{{NH}_{{3}_{ppm ( i )}}}{{Total}_{ppm ( i )}} \times 100 \qquad [ Equation 3 ]$$

$$i = Data Number , {NH}_{{3}_{Ratio}} = COMPONENT RATIO OF {NH}_{3}$$

$${NH}_{{3}_{ppm}} = MEASURED CONCENTRATION OF {NH}_{3}$$

$${Total}_{ppm} = TOTAL SUM OF GAS CONCENTRATIONS MEASURED BY ELEVEN SENSORS$$

$${Sensor}_{Ratio} ( i ) [ \% ] = \frac{{Sensor}_{ppm} ( i )}{{noNH}_{3} {\_Total}_{ppm ( i )}} \times 100 \qquad [ Equation 4 ]$$

$$i = Data Number , {sensor}_{Ratio} = COMPONENT RATIOS OF THE OTHER GASES EXCLUDING {NH}_{3}$$

$${sensor}_{ppm} = MEASURED CONCENTRATIONS OF THE OTHER GASES EXCLUDING {NH}_{3} ,$$

$${noNH}_{3} {\_Total}_{ppm} = MEASURED CONCENTRATIONS OF THE OTEHR TEN GASES EXCLUDING {NH}_{3}$$

According to an embodiment of the present disclosure, the component ratio value in the process (c) may be obtained by taking a single data value from the real-time component ratio graph for each gas derived in the process (b), or by selecting a certain range and calculating an average value within the range. However, the present disclosure is not limited thereto. The range for calculating the average value may vary depending on the data analysis conditions.

According to an embodiment of the present disclosure, the graph in the process (d) refers to any type of graph that can be expressed in a graphical form, such as a bar graph, line graph, pie chart, or band graph, and is not limited to any specific type of graph.

Hereinafter, the present disclosure will be explained in more detail with reference to Examples. However, the following Examples are illustrative only for better understanding of the present disclosure, but do not limit the present disclosure.

### MODE FOR CARRYING OUT THE INVENTION

### Examples

### Example 1. Gas Sensor Setup and Sample Placement

### (1) Preparation of Gas Sensor

Gas sensors were connected to a power supply for stabilization. Then, a gas sensor array was configured to measure human breath in order to verify that the gas sensors were properly connected and functioning normally.

### (2) Preparation and Placement of Sample

After a stool collection paper was placed in a toilet, a switch was turned on to begin data acquisition from the gas sensors. After two minutes, a fan was turned on to allow outside air to be drawn into and circulated through the gas sensor array. After another two minutes, a prepared stool sample was placed on the stool collection paper, and a toilet lid was closed to create a sealed environment. A tube was attached to the toilet lid to connect the toilet to the gas sensor array, thereby allowing fecal gases from the toilet to be drawn into the sensor array. The sensors used for component ratio analysis included CO, H₂, NH₃, VOC, NO, NO₂, H₂S, SO₂, C₂H₄, CH₂O and ETO sensors. Fecal gases were measured for about 3 minutes to 30 minutes. Then, the tube was disconnected. After two minutes, the switch of the gas sensors was turned off to stop data transmission.

### (3) Determination of Response Status of Each Sensor

When the fecal gases were measured, a response of each sensor was examined. For any sensor determined to show no response, all measured values over time were adjusted to 0 ppm. This is because if the measured values are not adjusted to 0 ppm, the sensor's noise is read as a measured value, which affects the component ratio analysis. The criterion for determining whether a sensor responded was as follows. When a signal value obtained during fecal gas measurement for each sensor was greater than 0.8 times the maximum signal noise value recorded for that sensor before fecal gas measurement, the sensor was considered responsive.

(Maximum signal noise value before fecal gas measurement for each sensor)×0.8<(Signal magnitude during fecal gas measurement)

### Example 2. Real-Time Component Ratio Graph Analysis

### (1) Output of Real-Time Component Ratio Graph

After it was determined whether each sensor responded to fecal gases, a real-time component ratio graph was derived as shown below. In the test described below, the NH₃ sensor was used as a reference sensor (i.e., ex_sensor=NH₃).

When the component ratio for the NH₃ sensor (ex_sensor in Equation 1 below) was calculated, a real-time component ratio graph for the NH₃ sensor was output based on a total gas concentration graph obtained by summing the measurement values from all sensors (including the NH₃ sensor) in a sensor set, as shown in Equation 1 below. To output the real-time component ratio for the NH₃ sensor, the total gas concentration (Total_ppm) was calculated by summing the real-time measurement values from all eleven sensors (non-responsive sensors set to 0 ppm) (see Equation 1).

$${ex\_sensor}_{Ratio} ( i ) [ \% ] = \frac{{ex\_sensor}_{ppm ( i )}}{{Total}_{ppm ( i )}} \times 100 \qquad [ Equation 1 ]$$

$$i = Data Number , {ex\_sensor}_{Ratio} = \begin{matrix}COMPONENT RATIO MEASURED BY A SPECIFIC SENSOR \\ FOR SEPARATE ANALYSIS\end{matrix}$$

$${ex\_sensor}_{ppm} = CONCENTRATION MEASURED BY A SPECIFIC SENSOR FOR SEPARATE ANALYSIS ,$$

$${Total}_{ppm} = TOTAL SUM OF GAS CONCENTRATIONS MEASURED BY ELEVEN SENSORS$$

When the component ratios obtained from the sensors other than the NH₃ sensor were calculated, a “total concentration graph [ppm] for sensor set excluding NH₃” was derived and used to generate the component ratio graphs for the other sensors in order to minimize the influence of the NH₃ sensor on the other sensors. For the other sensors, the component ratios were calculated based on no_ex_sensor_Total ppm (in this case, no_NH₃_Total ppm) obtained by summing the real-time measurement values from the ten sensors excluding the NH₃ sensor (non-responsive sensors set to 0 ppm) (see Equation 2).

$${Sensor}_{Ratio} ( i ) [ \% ] = \frac{{Sensor}_{ppm} ( i )}{no\_ex \_sensor {\_Total}_{ppm ( i )}} \times 100 \qquad [ Equation 2 ]$$

$$i = Data Number , {sensor}_{Ratio} = \begin{matrix}COMPONENT RATIOS OF GASES MEASURED BY \\ THE OTHER TEN SENSORS EXCLUDING ex\_sensor\end{matrix}$$

$${sensor}_{ppm} = GAS CONCENTRATIONS MEASURED BY THE OTHER SENSORS EXCLUDING ex\_sensor ,$$

$$no\_ex \_sensor {\_Total}_{ppm} = \begin{matrix}TOTAL SUM OF GAS CONCENTRATIONS MEASURED BY \\ THE OTHER TEN SENSORS EXCLUDING ex\_sensor\end{matrix}$$

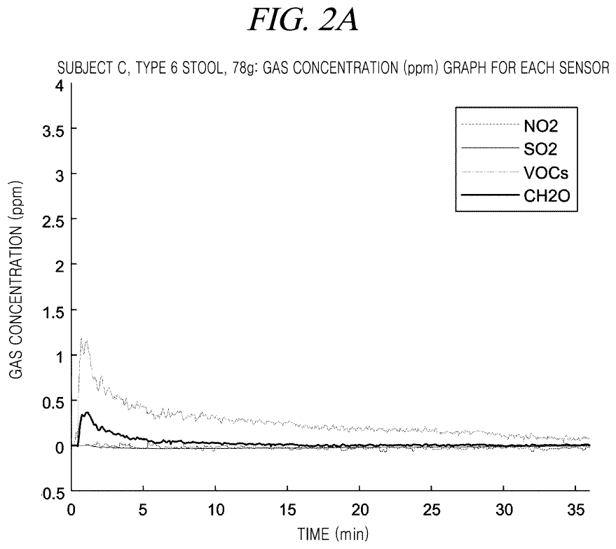

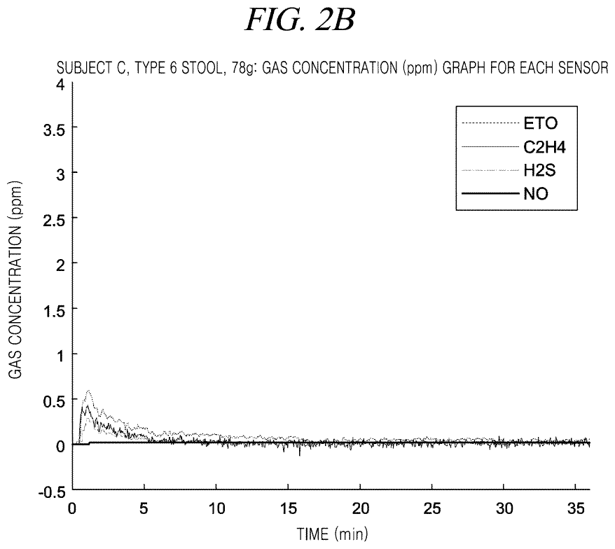

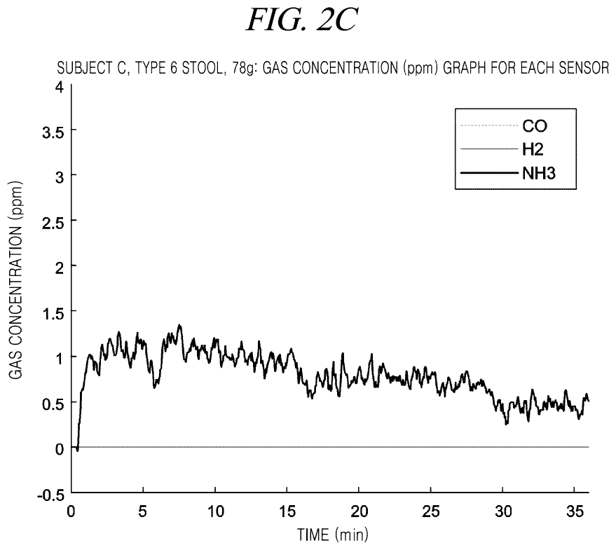

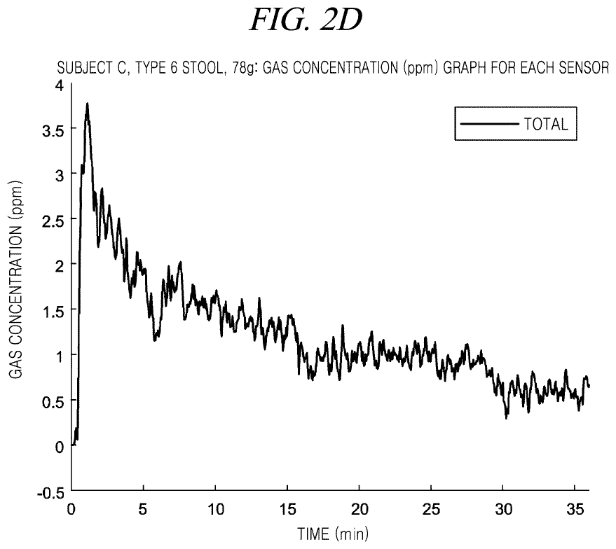

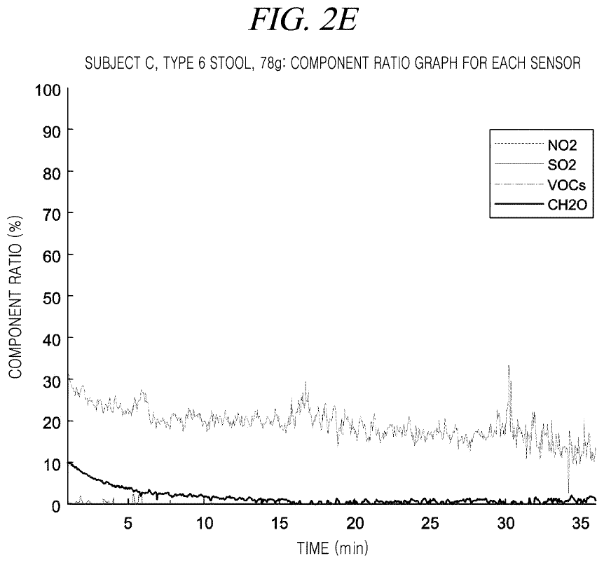

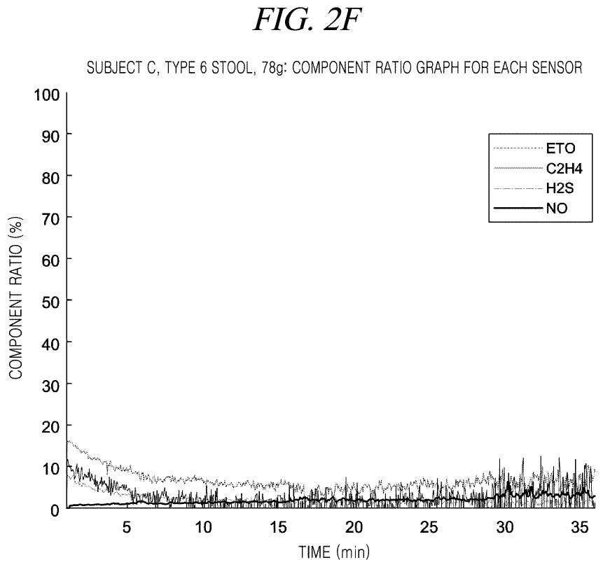

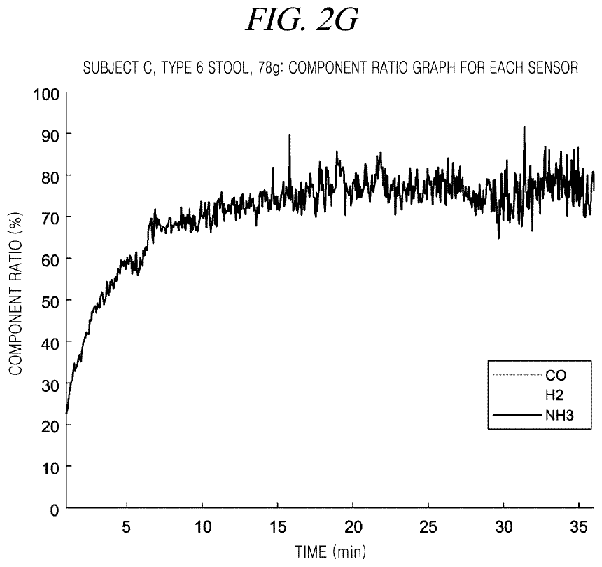

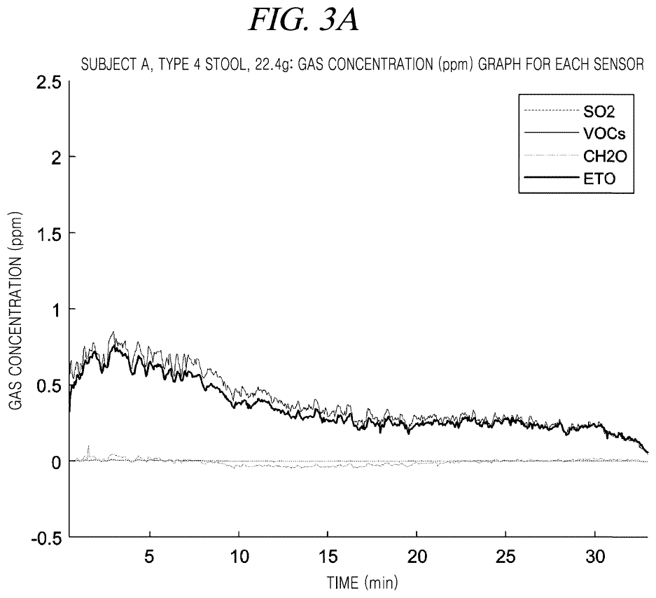

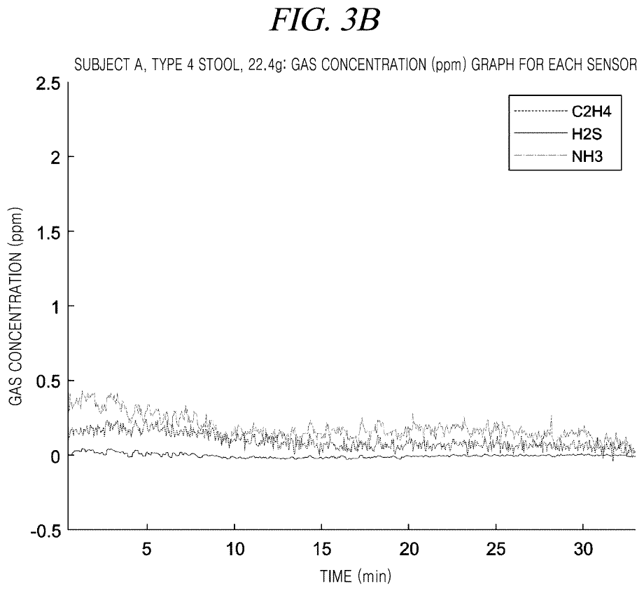

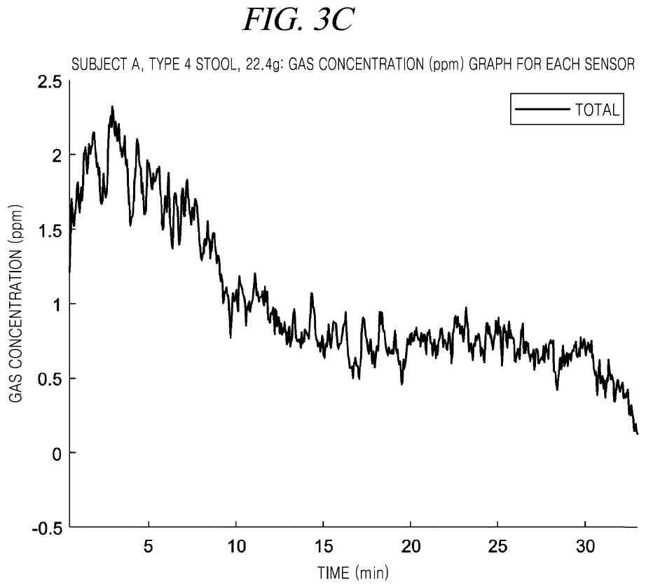

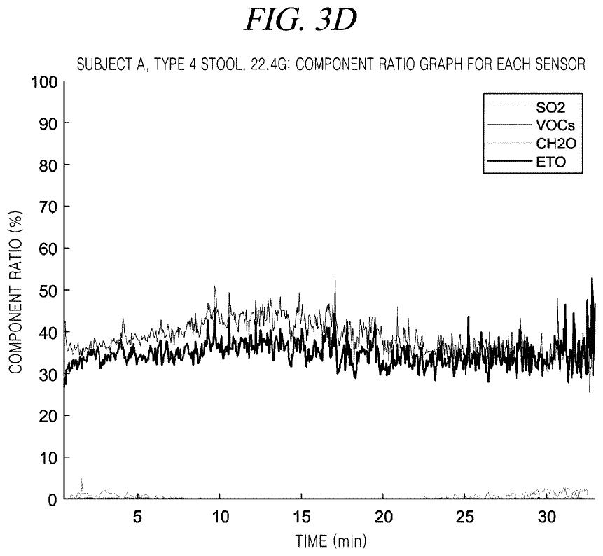

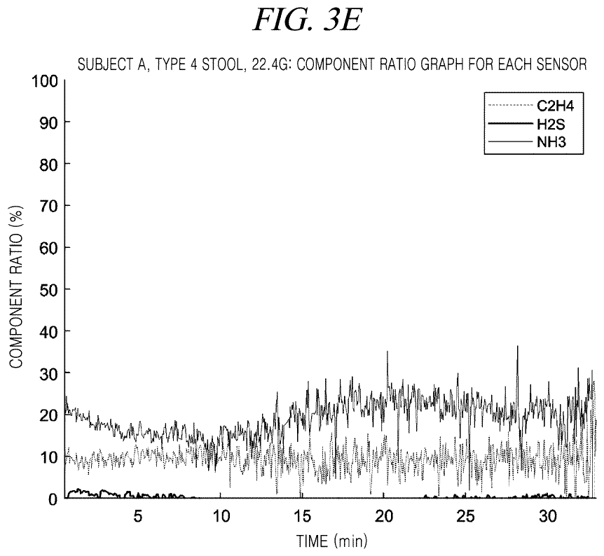

In the gas concentration graphs shown in FIG. 2A to FIG. 2D and FIG. 3A to FIG. 3C, the measurement values from the sensors continuously decreased over time throughout the entire duration of fecal gas measurement. However, as shown in FIG. 2E to FIG. 2G and FIG. 3D and FIG. 3E, the component ratio graph for each sensor maintained almost constant values after a certain point in time (approximately 10 minutes in FIG. 2E to FIG. 2G), showing little or no decrease over time. Further, it was confirmed that even when the sensor set used for component ratio analysis in FIG. 2A to FIG. 2D and FIG. 2E to FIG. 2G was changed, the component ratio values remained constant over time.

### Meaning of “the Component Ratio in the Real-Time Component Ratio Graph Remains Constant Over Time”

“The values do not consistently decrease or increase over time until the stool sample is removed (during the stool measurement period).” In the component ratio graphs shown in FIG. 3D and FIG. 3E, although the signals from the NH₃ sensor exhibited slight fluctuations, there was no trend of continuous decrease or increase over time, which indicates that these variations were not caused by the passage of time. The minor signal fluctuations observed in the sensor are presumed to result from environmental influences, such as air flow, during a relatively long measurement duration of about 30 minutes.

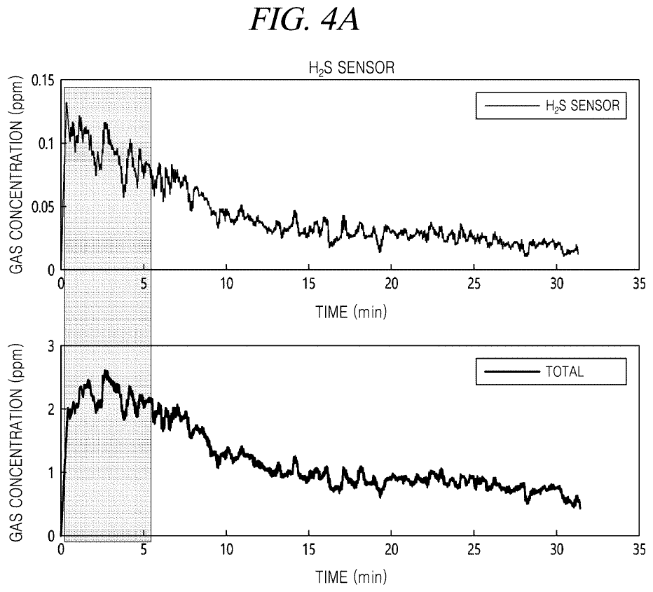

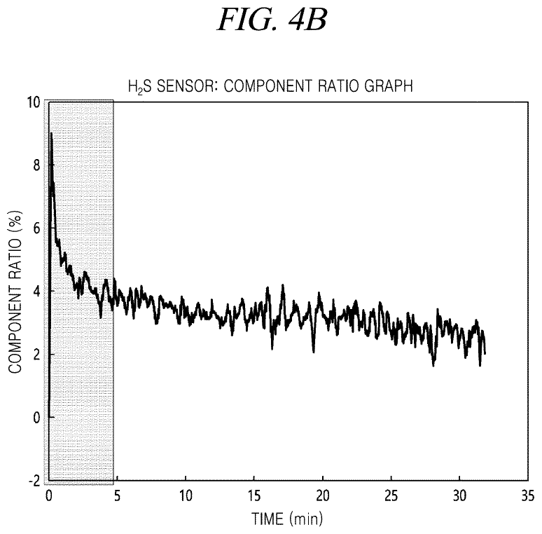

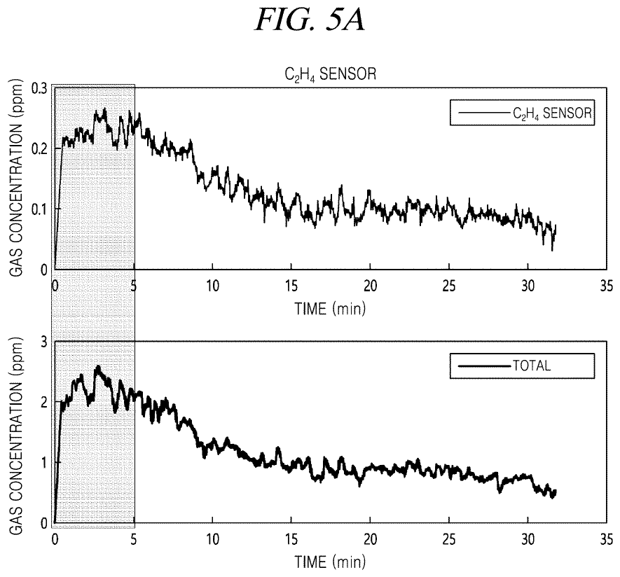

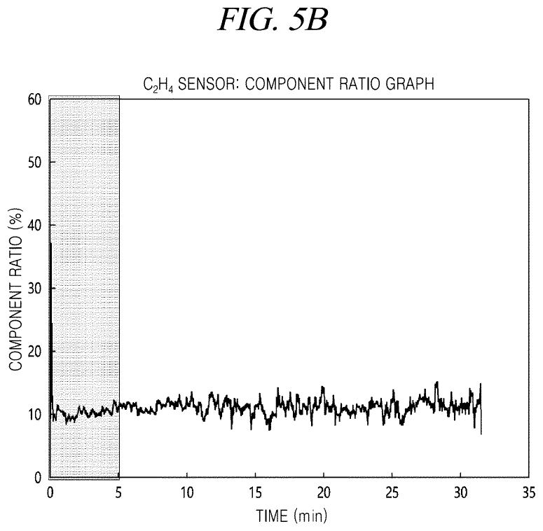

In FIG. 2E to FIG. 2G, the component ratios obtained from some sensors decreased during the initial five-minute measurement period. However, since the fecal gas concentration (ppm) decreased sharply only during the initial measurement period and not throughout the entire 30-minute measurement period, this behavior was attributed not to the passage of time but to differences in the rate and slope of decrease between the “concentration graph (ppm) for one sensor” and the “total concentration graph (ppm)” obtained by summing the measurement values from the sensor set. For example, as shown in FIG. 4A, the concentration (ppm) measured by the H₂S sensor exhibited a steeper decrease than the total concentration graph (ppm) for the sensor set, which causes an early decrease observed in the component ratio graph shown in FIG. 4B. Meanwhile, the concentration (ppm) measured by the C₂H₄ sensor decreased at nearly the same rate as the total concentration graph (ppm) for the sensor set. Therefore, no significant change in the component ratio value was observed in the initial stage of the real-time component ratio graph shown in FIG. 5B.

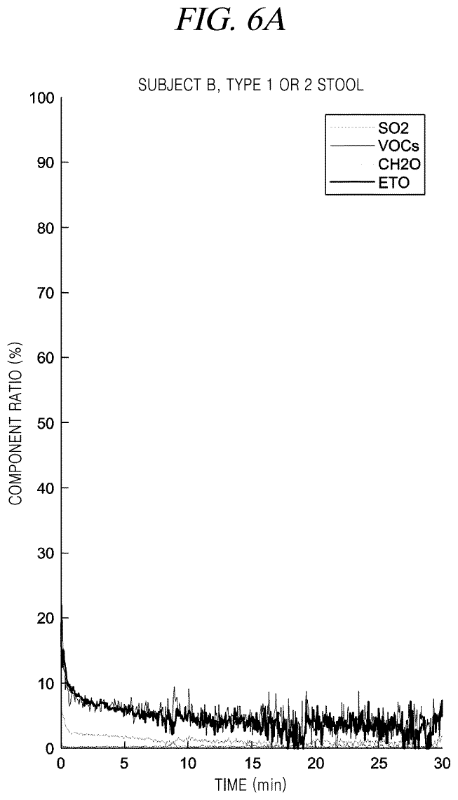

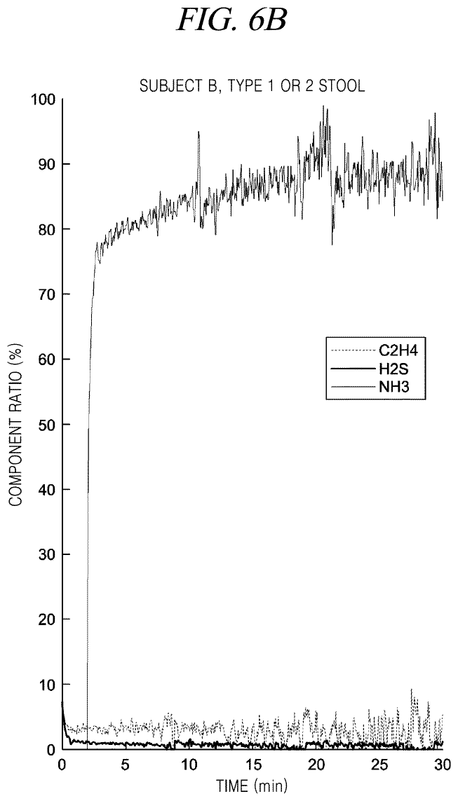

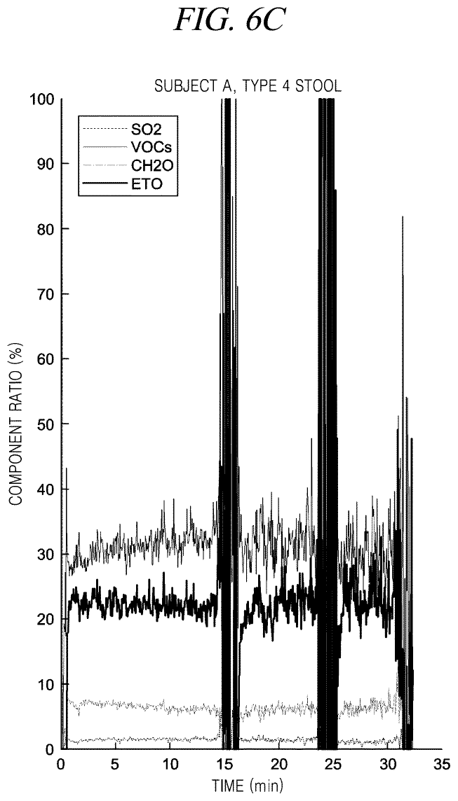

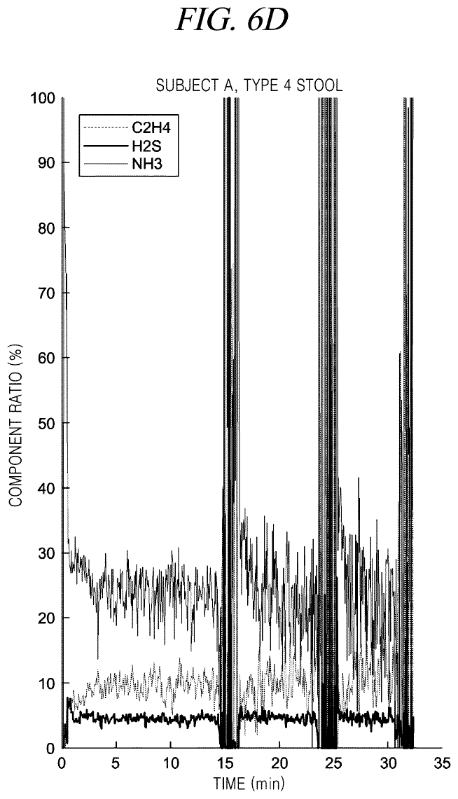

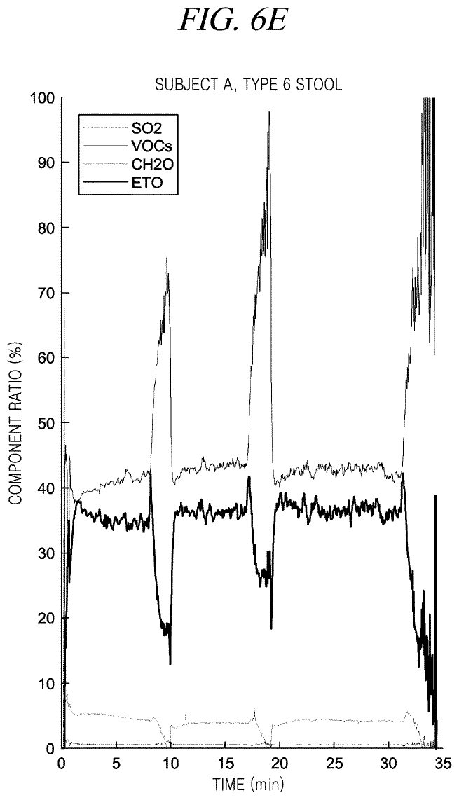

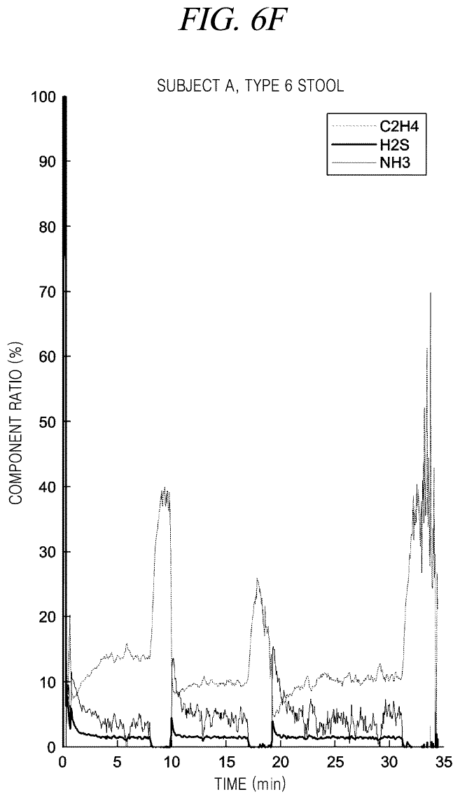

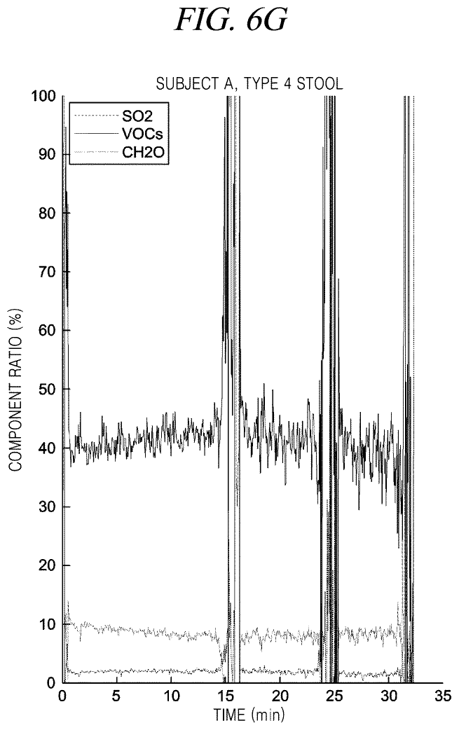

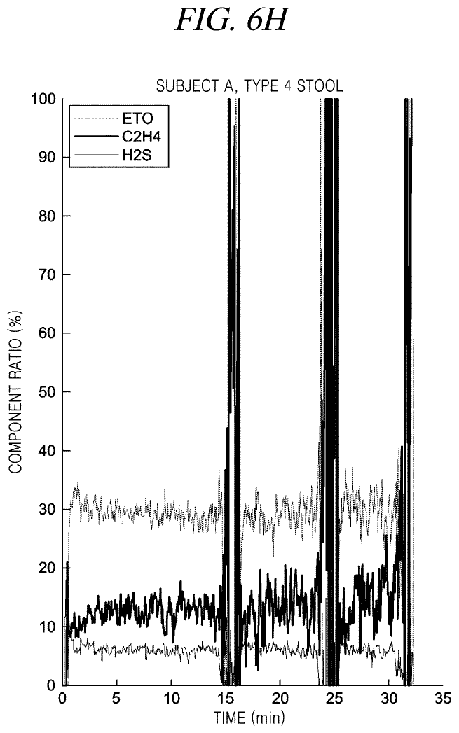

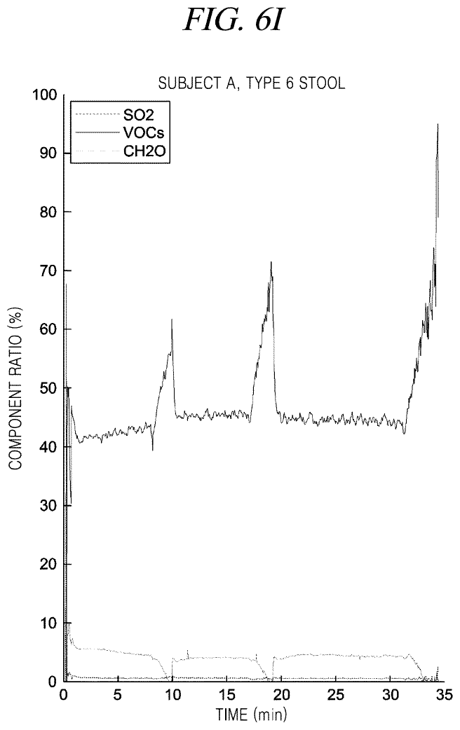

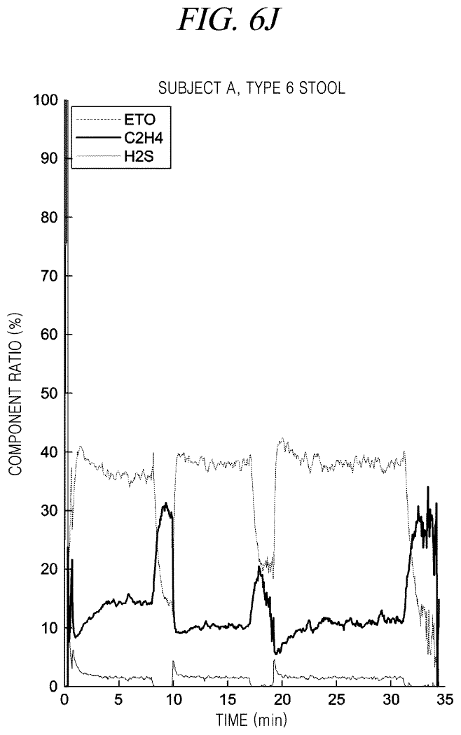

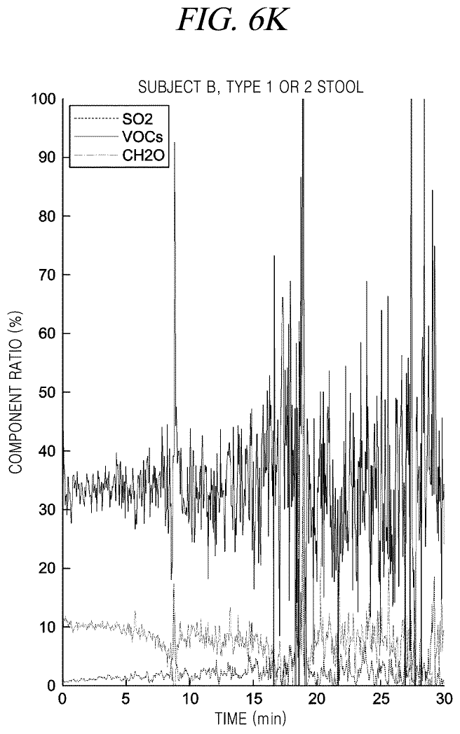

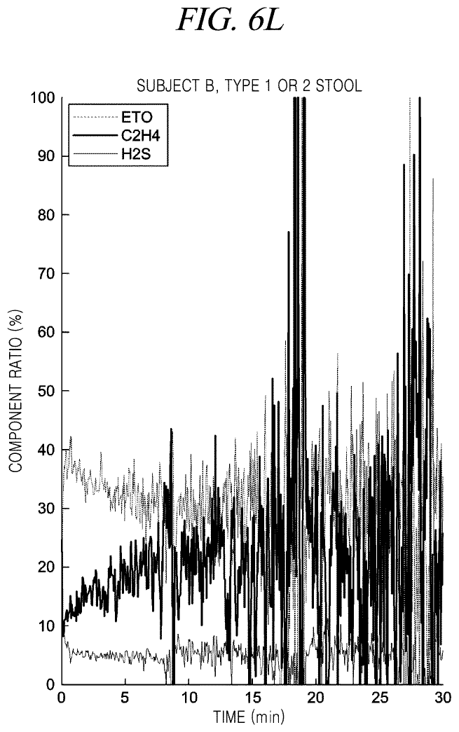

Referring to FIG. 6A to FIG. 6F, as the stool type changed from Type 2 toward Type 6, the component ratios obtained from the sensors, particularly from the NH₃ sensor, were changed markedly. This confirmed that a subject's health condition can be assessed using the component ratio analysis method. In the real-time component ratio graphs of fecal gases for Type 4 and Type 6 stool, the sensor signals exhibited spikes in sections where fecal gas measurement was intermittently stopped and resumed during the test. Therefore, these sections were excluded from component ratio analysis.

TABLE 1

| | | Relative magnitude relationship |
|---|---|---|
| Stool | | of component ratios among seven |
| type | Stool | sensors |
| 2Type | Subject B 1~2Type 43 g | NH₃ > VOCs > ETO > C₂H₄ > |
| | | CH₂O > H₂S > SO₂ |
| | Subject B 2Type 55 g | NH₃ > VOCs > ETO > C₂H₄ > |
| | | CH₂O > H₂S > SO₂ |
| 4Type | Subject A 4Type 47 g | VOCs > ETO > C₂H₄ > NH₃ > |
| | | CH₂O > H₂S > SO₂ |
| | Subject A 4Type 27.4 g | VOCs > NH₃ > ETO > C₂H₄ > |
| | | CH₂O > H₂S > SO₂ |
| | Subject A 4Type 22.7 g | VOCs > ETO > NH₃ > C₂H₄ > |
| | | CH₂O > H₂S > SO₂ |
| | Subject A 4Type 20.67 g | VOCs > ETO > NH₃ > C₂H₄ > |
| | | CH₂O > H₂S > SO₂ |
| 6Type | Subject C 6Type 23.01 g | VOCs > ETO > C₂H₄ > CH₂O > |
| | (After consumption of | H₂S > NH₃ > SO₂ |
| | spicy food) | |
| | Subject C 6Type 110.06 g | VOCs > ETO > C₂H₄ > NH₃ > |
| | | CH₂O > H₂S > SO₂ |

As shown in Table 1, in all measured stool samples, the relationship of component ratios obtained from the sensors excluding the NH₃ sensor remained unchanged. However, the NH₃ sensor showed variations in its relative position with respect to the component ratios for each stool sample. In FIG. 6A to FIG. 6F, the NH₃ sensor exhibited component ratio values exceeding about 90% as the stool type approached Type 1 or 2, whereas the component ratio values dropped below 10% as the stool type approached Type 6. Since the NH₃ sensor values varied significantly with stool type, the component ratios obtained from the other sensors were more greatly influenced by the NH₃ component ratio than by the stool type. Accordingly, in this study, the NH₃ sensor among the eleven-sensor set was classified as a specific sensor, and the component ratios from the other sensors were reanalyzed after excluding the specific sensor (NH₃ sensor) to examine trends and characteristics associated with stool type and the subject's health condition. The component ratio analysis can also be applied to any sensor combination composed of a plurality of sensors (a partial combination of the eleven sensors or other sensor combinations not mentioned herein) by selecting and classifying a specific sensor.

Meanwhile, as shown in FIG. 6G to FIG. 6L, it was difficult to analyze trends or distinguish characteristics of different stool types using the real-time component ratio graphs for the sensors excluding the NH₃ sensor. Therefore, the present inventors quantified the previously analyzed real-time component ratio graphs, summarized the component ratio values from the respective sensors included in the sensor set for a single fecal gas sample, and represented the results as bar graphs for analysis.

### Example 3. Analysis of Real-Time Component Ratio Bar Graph

The real-time component ratio bar graphs were analyzed through the following processes.

### Process 1: Collection of Stool Data

As shown in Table 2 below, data from 18 stool samples, each collected on different days from three subjects, were gathered for fecal gas component ratio analysis. The collected stool samples varied in Bristol stool type, diet, subject, gender, age, and stool weight.

TABLE 2

| Subject | Stool type | |
|---|---|---|
| Subject A (Female) | 1 | 3~4Type |
| Normal stool type: 4Type | 2 | 4Type |
| | 3 | 4Type |
| | 4 | 4Type |
| | 5 | 4Type |
| | 6 | 6Type |
| | 7 | 6Type |
| | 8 | 6Type |
| | 9 | 6Type |
| | 10 | 6Type |
| Subject B (Female) | 1 | 2Type |
| Normal stool type: 2Type | 2 | 2Type |
| | 3 | 3Type |
| | 4 | 3Type |
| | 5 | 3Type |
| | 6 | 3Type |
| Subject C (Male) | 1 | 4Type |
| Normal stool type: 4Type | 2 | 6Type |

### Process 2: Derivation of Real-Time Component Ratio Graph for Each Stool Sample, Quantification of Component Ratio and Conversion to Bar Graph

### Process 3: Bar Graph Analysis

When 18 stool samples collected from the three subjects were analyzed using the eleven-sensor set, the NO₂ and NH₃ sensors exhibited a trend in which their component ratio values gradually decreased as the stool type became more diarrheal, i.e., toward Types 6 to 7. Further, when only the stool data of subject A were analyzed with the CH₂O sensor, Types 6 to 7 were distinguishable from Types 3 to 4.

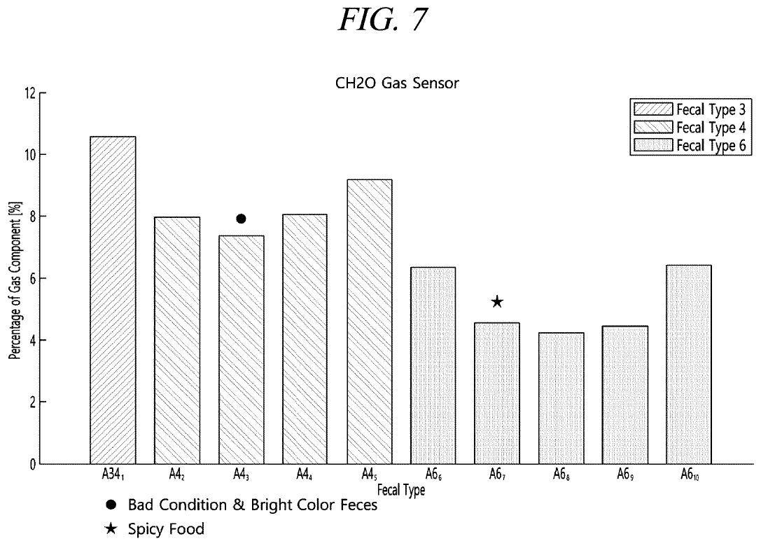

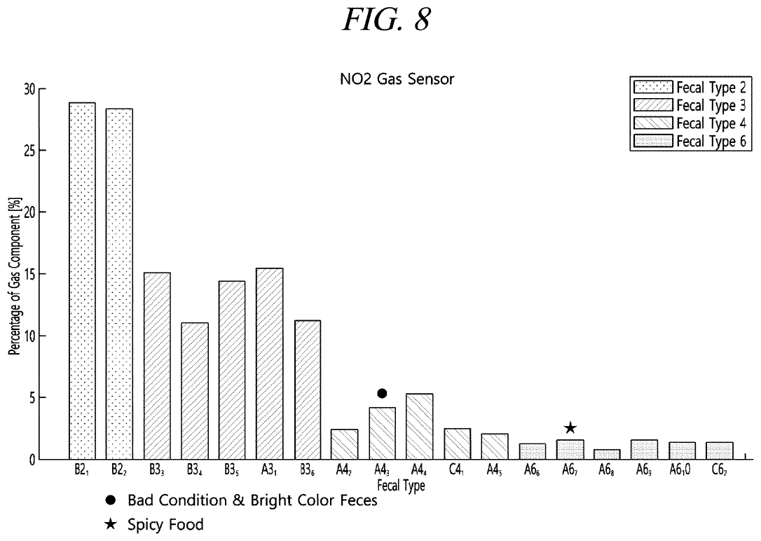

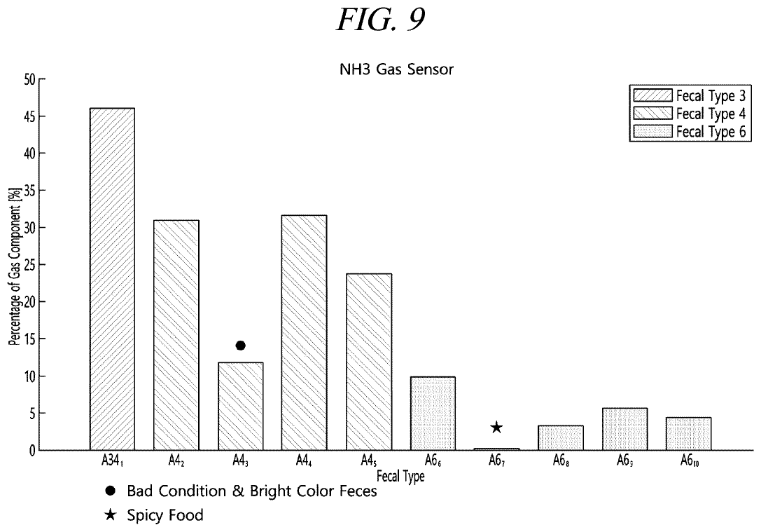

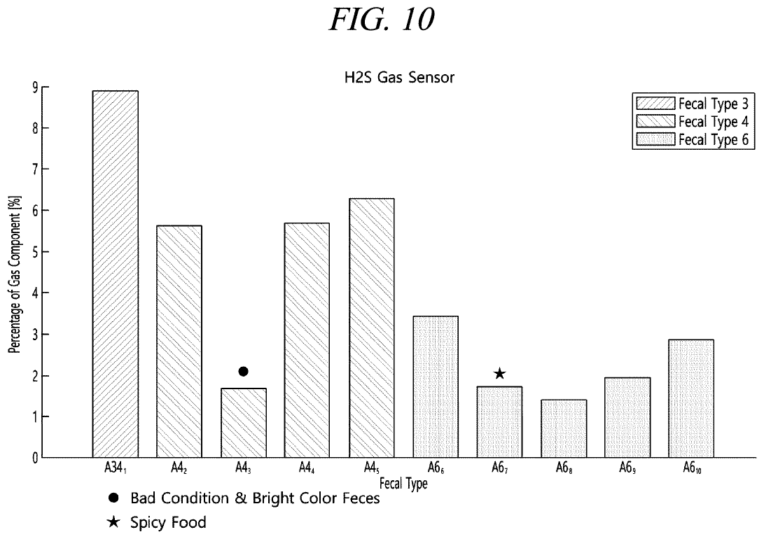

When only the data of subject A were analyzed, it become easier to determine the Bristol stool type and health condition. In the bar graph for the CH₂O sensor, Types 3 to 4 and Types 6 to 7 could be distinguished (see FIG. 7). In the bar graph for the H₂S sensor, although the stool was Type 4, which is considered a healthy stool type, additional detailed information was obtained, such as the stool being light in color and associated with digestive discomfort (see FIG. 10). Also, in the bar graph for the NH₃ sensor, the Type 4 stool was light in color and associated with digestive discomfort, and the Type 6 stool was identified as obtained after consumption of spicy food (see FIG. 9). These findings demonstrate that, through analysis of component ratios obtained from eleven sensors, both the Bristol stool type and the health condition can be effectively distinguished and identified.

In conclusion, it was confirmed that the fecal gas component ratio analysis method of the present disclosure enables classification of Bristol stool types or identification of an individual's health condition regardless of fecal gas measurement time or stool weight.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description. It shall be understood that all modifications and embodiments conceived from the meaning and scope of the claims and their equivalents are included in the scope of the present disclosure.

We claim:

1. A fecal gas component ratio analysis method for Bristol stool type classification and body condition analysis, comprising:
a process (a) of measuring a fecal gas concentration value (ppm) using a gas sensor;
a process (b) of deriving a real-time component ratio graph for each gas based on the measured gas concentration value;
a process (c) of obtaining a component ratio value from the derived real-time component ratio graph;
a process (d) of deriving a graph using the obtained component ratio value; and
a process (e) of classifying a Bristol stool type of feces by comparing the derived graph with a component ratio graph for each Bristol stool type.

2. The fecal gas component ratio analysis method of claim 1,
wherein the gas sensor used in the process (a) includes one or more selected from the group consisting of NH₃, CO, H₂, VOC, NO, NO₂, H₂S, SO₂, C₂H₄, CH₂O and ETO gas sensors.

3. The fecal gas component ratio analysis method of claim 1,
wherein the process (b) is performed as shown in Equations 1 and 2 below:
$${ex\_sensor}_{Ratio} ( i ) [ \% ] = \frac{{ex\_sensor}_{ppm ( i )}}{{Total}_{ppm ( i )}} \times 100 \qquad [ Equation 1 ]$$
$$i = Data Number , {ex\_sensor}_{Ratio} = \begin{matrix}COMPONENT RATIO MEASURED BY A SPECIFIC SENSOR \\ FOR SEPARATE ANALYSIS\end{matrix}$$
$${ex\_sensor}_{ppm} = CONCENTRATION MEASURED BY A SPECIFIC SENSOR FOR SEPARATE ANALYSIS ,$$
$${Total}_{ppm} = TOTAL SUM OF GAS CONCENTRATIONS MEASURED BY ELEVEN SENSORS ,$$
$$and$$
$${Sensor}_{Ratio} ( i ) [ \% ] = \frac{{Sensor}_{ppm} ( i )}{no\_ex \_sensor {\_Total}_{ppm ( i )}} \times 100 \qquad [ Equation 2 ]$$
$$i = Data Number , {sensor}_{Ratio} = \begin{matrix}COMPONENT RATIOS OF GASES MEASURED BY \\ THE OTHER TEN SENSORS EXCLUDING ex\_sensor\end{matrix}$$
$${sensor}_{ppm} = GAS CONCENTRATIONS MEASURED BY THE OTHER SENSORS EXCLUDING ex\_sensor ,$$
$$no\_ex \_sensor {\_Total}_{ppm} = \begin{matrix}TOTAL SUM OF GAS CONCENTRATIONS MEASURED BY \\ THE OTHER TEN SENSORS EXCLUDING ex\_sensor\end{matrix}$$
